(19) United States
(12) Patent Application Publication
(10) Pub. No.: US 2010/0152564 A1
(43) Pub. Date: Jun. 17, 2010

(54) Method and apparatus for external monitoring device

(76) Inventors: Hoang Van Nguyen, San Jose, CA (US); Ngoc Anh Mai, San Jose, CA (US); Nu Ngo, San Jose, CA (US); Kannon Do, San Jose, CA (US)

Correspondence Address: HOANG VAN NGUYEN, 1273 ALVERNAZ DR., SAN JOSE, CA, 95121, US

(21) Appl. No.: 12/316,723

(22) Filed: Dec. 16, 2008

(51) Int. Cl. A61B 5/04

(52) U.S. Cl. 600/390

(57) ABSTRACT

A monitoring device is disclosed that includes a sensor, a belt, and a cushion pad. The sensor includes a front side and a back side, the front side being placed directly onto the body of a patient so as to acquire medical vital signals from within the body of said patient, the back side having a connector pin. The belt is operable to hold the sensor to a fixed location on the body. The cushion pad is positioned between the sensor and the belt so as to press the sensor close to the body of said patient without having to tightening the belt.

### FIELD OF THE INVENTION

The present invention relates generally to the field of medical devices. More particularly, the present invention relates to the field of monitoring vital signals from within a human body using external electronic devices.

### BACKGROUND

Monitoring vital signals from within a human body is important and essential during clinical treatment and examination of a patient. For example, many pregnant patients require continuous monitoring because of the medication they are receiving or health concerns related to themselves or their unborn baby. Unlike internal monitoring, external monitoring is a safe and common method used by healthcare provider to determine the fetus' well being and to access labor progress.

Now referring to FIG. 1, a prior art external fetal heart rate monitor device 100 is illustrated. External fetal heart rate monitor device 100 comprises a sensor 104 that has a back side 105 and a front side 106. Back side 105 is connected to a connector pin 107 and front side 106 is positioned directly to the abdomen of a pregnant patient 101 as shown in FIG. 1. External fetal heart rate monitor device 100 also includes a belt 102 having a plurality of holes 103 lined up in a straight line. Sensor 104 is electrically connected to an electronic display device 110 such as an Electronic Fetal Monitor (EFM) via a cable 108 and a connector 109.

As can be seen from FIG. 1, after the position of the fetus is located by a nurse, sensor 104 is attached the abdomen of patient 101 with belt 102. More specifically, connector pin 107 is inserted in one of holes 103 and then belt 102 is wrapped around the abdomen of patient 101. Thereafter, connector pin 107 is again inserted into the same hole 103 to hold sensor 104 in place where the fetus is best located using known medical procedures. Cable 108 is connected between sensor 104 and electronic display unit 110. Often gel is applied to patient 101 to increase the conductivity of sensor 104. However, prior art fetal monitoring device 100 cannot adapt to different physical conditions of patient 101 and therefore the heart rate signals of the fetus cannot be effectively picked up by electronic display unit 110. One of such physical condition is the layers of fat underneath the abdomen of patient 101. In other circumstances, it is difficult to pick up heart beat of signals of the infant of pre-term pregnant women. This is because the heat beat of pre-term infants are too weak. To overcome these problems, nurses or healthcare providers tightens prior-art belt 102 further by connecting connector pin 107 to further hole 103 to the right of patient 101 so as to press sensor 104 closer to the fetus. This method causes intolerable pains and extreme discomforts to patient 101.

Thus, there is a need for an external monitoring device such as the fetal heart rate monitor that can adapt to different physical and medical conditions of a patient and effectively monitor the heart rate signals from a fetus without causing pains and discomforts to the patient. In addition, there is a need for a low cost and simple external monitor device that can be easily used by a nurse or by a patient herself.

### SUMMARY OF THE INVENTION

A monitoring device is disclosed that includes a sensor, a belt, and a cushion pad. The sensor includes a front side and a back side, the front side being placed directly onto a body of a patient so as to acquire medical vital signals from within the body of said patient, the back side having a connector pin. The belt is operable to hold the sensor to a fixed location on the body. The cushion pad is positioned between the sensor and the belt so as to press the sensor close to the body of said patient without having to tightening the belt, thus avoiding causing pains to the patient.

A method of monitoring vital signals from a human body is also disclosed that includes the steps of placing a sensor on the body of a patient, determining whether vital signals are received, whenever vitals signals are not received, placing a cushion pad on the other side of the sensor; and adjusting the thickness of the cushion pad until the vitals signals are detected.

The above and still further objects, features and advantages of the present invention will become apparent upon consideration of the following detailed description of several specific embodiments thereof, especially when taken in conjunction with the accompanying drawings.

### BRIEF DESCRIPTION OF THE DRAWINGS

The accompanying drawings, which are incorporated in and form a part of this specification, illustrate embodiments of the invention and, together with the description, serve to explain the principles of the invention.

FIG. 1 illustrates a prior art external fetal heart rate monitoring device that is commonly used by healthcare providers and patients.

FIG. 2 illustrates different components of a monitoring device,including a sensor, a belt, and a cushion pad in accordance with an embodiment of the present invention.

FIG. 3 illustrates the structure of a multi-layered cushion pad whose thickness can be changed by removing layers in accordance with an embodiment with the present invention.

FIG. 4 illustrates the structure of a multi-layered cushion pad whose first and last layers are adhesive in accordance with another embodiment of the present invention

FIG. 5 illustrates a cushion pad whose thickness can be changed by an electrical motor in accordance with an embodiment with the present invention.

FIG. 6 illustrates a cushion pad whose thickness can be changed by mechanically twisting the two cylindrical portions together in accordance with an embodiment of the present invention.

FIG. 7 illustrates a cushion pad whose thickness can be changed by a hand pump in accordance with an embodiment of the present invention.

FIG. 8A illustrates a removable multi-layered cushion pad that has a side cut to slide into a connector pin so as to occupy a position between a sensor and a belt in accordance with an embodiment of the present invention.

FIG. 8B illustrates another shape of a removable multi-layered cushion pad that includes a side cut adapted to slide into a connector pin so as to occupy a position between a sensor and a belt in accordance with an embodiment of the present invention.

FIG. 9 illustrates the manner the monitor system of the present invention is connected together to monitor vital signals from a body of a patient in accordance with an embodiment of the present invention.

FIG. 10 illustrates a flow chart of a method of monitoring vital signals from a body of a patient in accordance with an embodiment of the present invention.

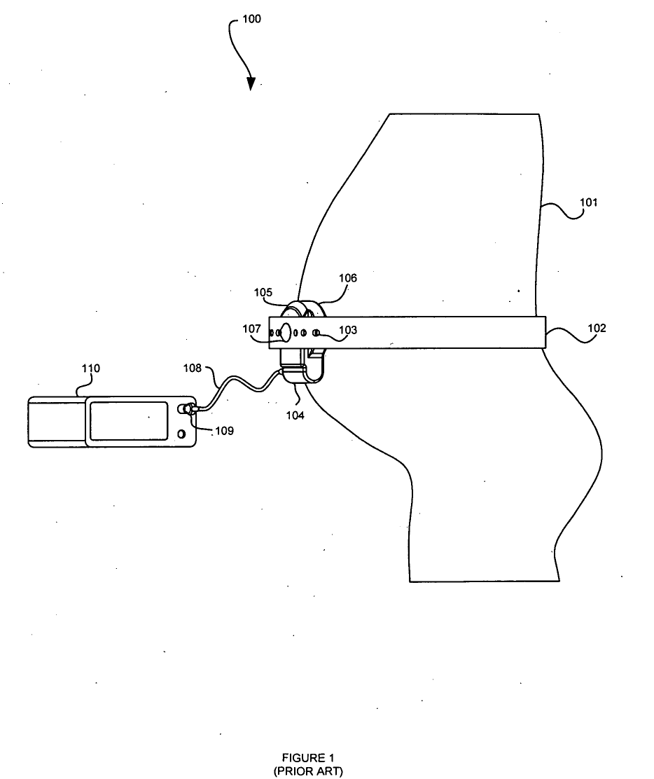

### DETAILED DESCRIPTION OF THE INVENTION

Reference will now be made in detail to different embodiments of the invention, examples of which are illustrated in the accompanying drawings. While the invention will be described in conjunction with the preferred embodiments, it will be understood that they are not intend to limit the invention to these embodiments. On the contrary, the invention is intended to cover alternatives, modifications and equivalents, which may be included within the spirit and scope of the invention as defined by the appended claims. Furthermore, in the following detailed description of the present invention, numerous specific details are set forth in order to provide a thorough understanding of the present invention. However, it will be obvious to one of the ordinary skill in the art that the present invention may be practiced without these specific details. In other instances, well-known methods, procedures, components, and circuits have not been described in detail so as not to unnecessarily obscure aspects of the present invention.

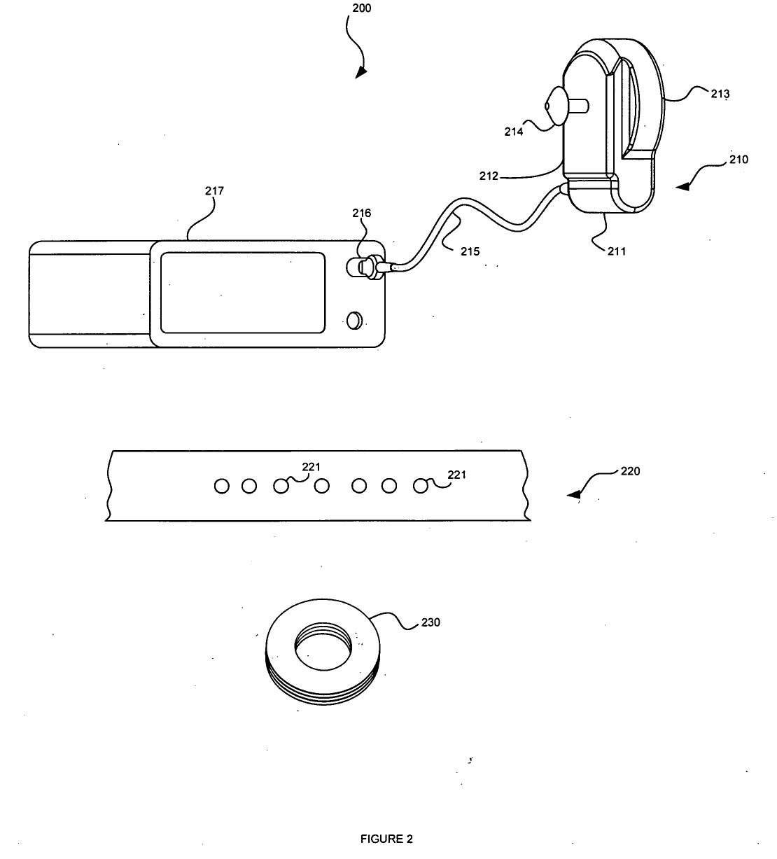

Referring to FIG. 2, various components of a monitor device 200 in accordance with an embodiment of the present invention are illustrated. More particularly, monitor device 200 includes an electronic section 210, a belt 220, and a cushion pad 230. Electronic section 210 further includes a sensor 211 which has a back side 212 and a front side 213. Back side 212 includes a connector pin 214 while front side 213 contacts the body of a patient where vital signals to be acquired. Sensor 211 is electrically connected to a cable 215 for transmitting vital signals to an electronic display unit 217. In one embodiment, cable 215 is also connected to an adapter 216. Belt 220 includes a plurality of holes 221 lined up in a straight line for fastening electronic section 210 onto the body of a patient. In one embodiment, belt 220 is elastic that can be stretched out to fit different physical sizes of different patients. Finally, cushion pad 230 in accordance with the present invention is used to press sensor 211 close to the body of a patient without stretching belt 220 and without having to couple connector pin 214 to one of holes 221 to the right of the patient, thus avoiding causing pains and discomforts to the patient. In an embodiment of the present invention, the thickness of cushion pad 230 can be adjustable to adapt to differences in physical sizes of different patients.

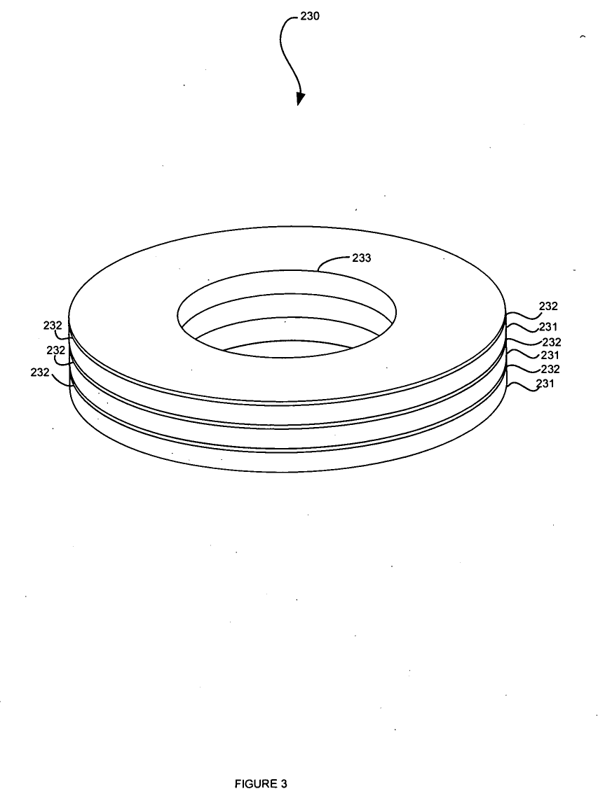

Referring next to FIG. 3, an embodiment of cushion pad 230 of the present invention is illustrated. Cushion pad 230 includes a plurality of first layers 231 interlaced with second layers 232. In one embodiment, second layer 232 is interposed between two adjacent first layers 231. In one embodiment, second layer 232 is non-adhesive so that first layer 231 is removable. In one embodiment, first layer 231 is made of cotton material while second layer 232 is made of plastic. In one embodiment, cushion pad 230 has a concentric opening 233 so that connector pin 214 of sensor 211 can be inserted through there.

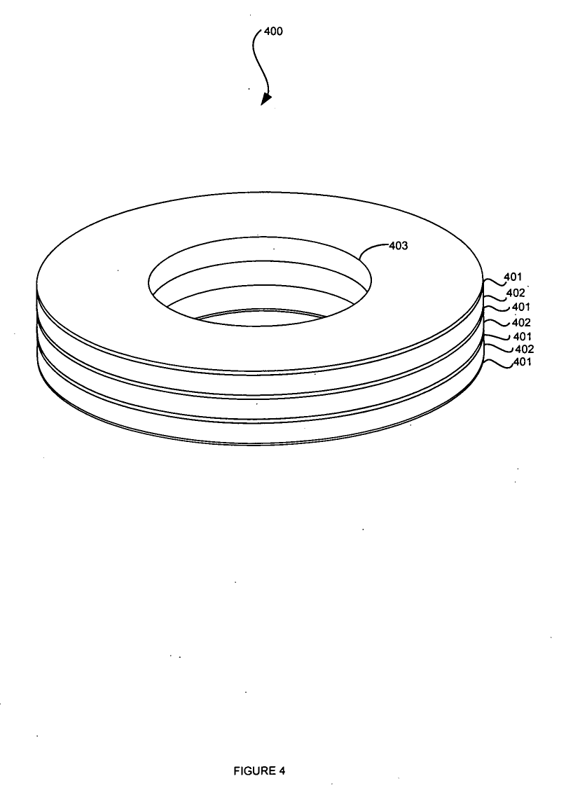

Referring next to FIG. 4, another embodiment of cushion pad 400 of the present invention is illustrated. Cushion pad 400 includes a plurality of first layers 401 interlaced with second layers 402. In one embodiment, second layer 402 is interposed between two adjacent first layers 401. In one embodiment, second layer 402 is non-adhesive so that first layer 401 is removable. In one embodiment, first layer 401 is made of cotton material while second layer 402 is made of plastic. In one embodiment, cushion pad 400 has a concentric opening 403 so that connector pin 214 of sensor 211 can be inserted there through. Cushion pad 400 is similar to cushion pad 230 shown in FIG. 3 and discussed above except that cushion pad 400 has first layers 401 and second layers 402 arranged so that the last layer is a first layer 401 which is adhesive. With such arrangement, cushion pad 400 is adapted to stick to backside 212 of sensor 211.

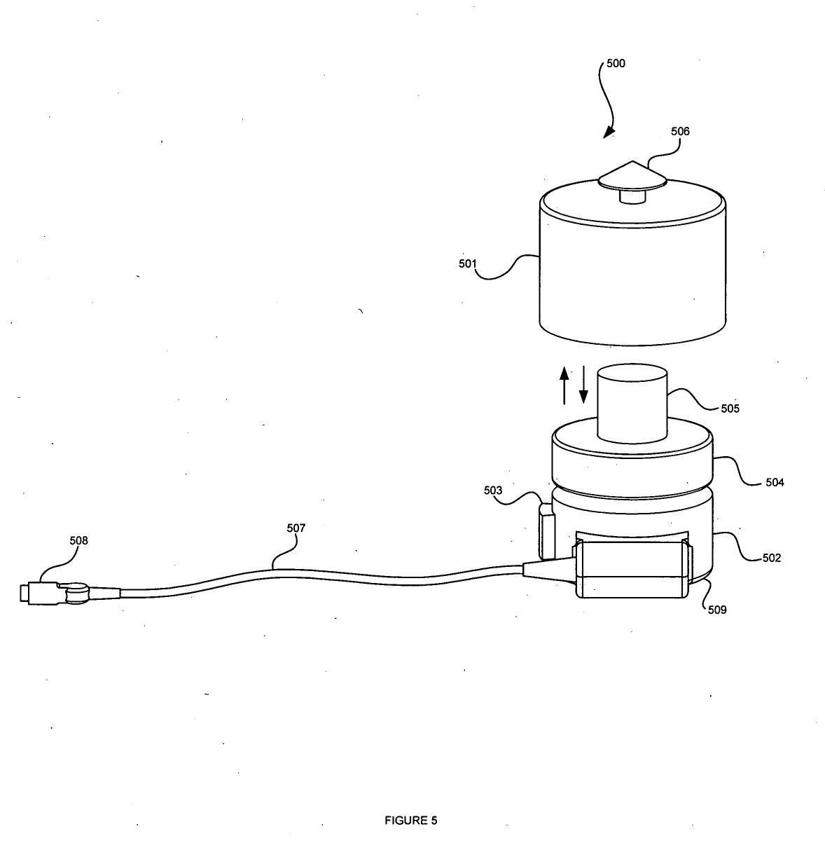

Referring next to FIG. 5, another embodiment of cushion pad 500 of the present invention is illustrated. More particularly, cushion pad 500 includes a first cylindrical portion 501 and a second cylindrical 502 portion. On the top side of first cylindrical portion 501, there is a connector pin 506 for coupling to belt 220. Second cylindrical portion 502 further includes an electrical motor 504 electrically and mechanically coupled to a switch 503. First cylindrical portion 501 is mechanically coupled to second cylindrical portion 502 via a center rod 505. In operation, when switch 503 is pressed by a healthcare provider or by the patient, electrical motor 504 pushes center rod 505 upward or downward so that first cylindrical portion 501 is pushed upward or downward respectively in relative to second cylindrical portion 502. Thus, the thickness of cushion pad 500 is adjustable depending on the physical condition of the patient without having to tighten belt 220 that may cause pains and discomforts to the patient. In one embodiment, cushion pad 500 is connected to a cable 507, an adaptor 508, and a sensor 509.

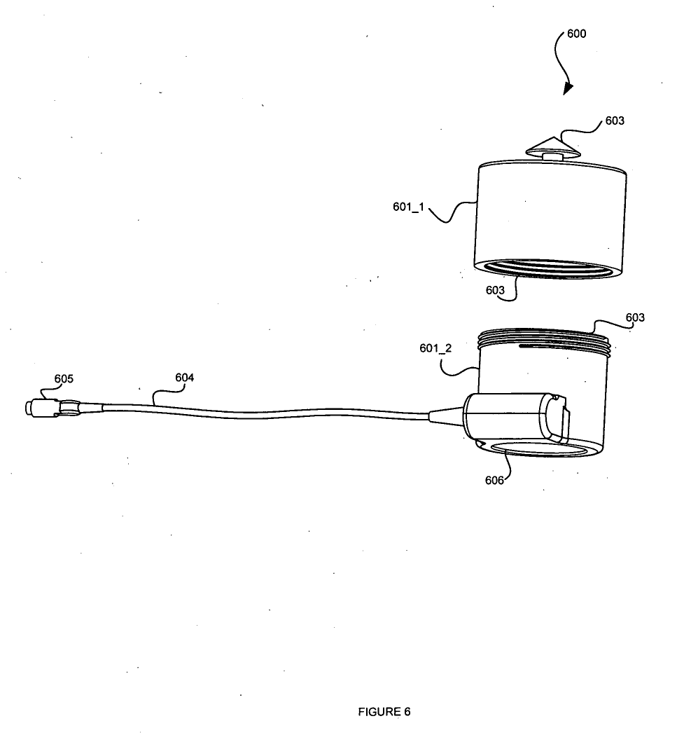

Referring next to FIG. 6, another embodiment of cushion pad 600 of the present invention is illustrated. More particularly, cushion pad 600 includes a first cylindrical portion 601_1 and a second cylindrical portion 601_2, both having threads 603 so that first cylindrical portion 601_1 can be mechanically twisted onto second cylindrical portion 601_2. As a result, the thickness of cushion pad 600 can be adjusted depending on the physical condition of the patient without having to tighten belt 220 that may cause pains and discomforts to the patient. In one embodiment, cushion pad 600 is connected to a cable 604, an adaptor 605, and a sensor 606.

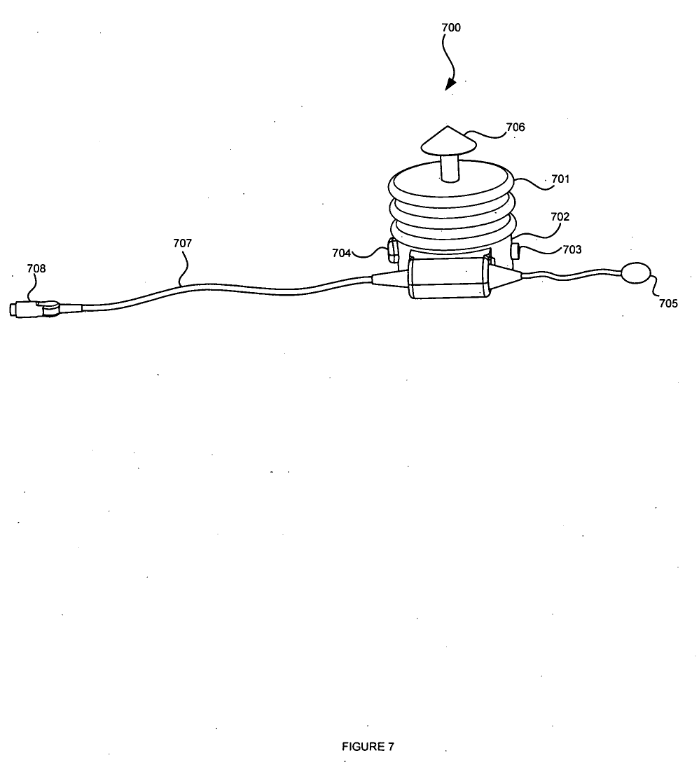

Referring next to FIG. 7, another embodiment of cushion pad 700 of the present invention is illustrated. More particularly, cushion pad 700 includes a main body 701 connected to a base 702 having a release port 703 and a release valve 704. Main body 704 is connected to a hand pump 705. As a result, the thickness of cushion pad 700 can be adjusted by manually pumping air into main body 701 using hand pump 705. After the monitoring has been complete, air can be released from main body 701 by pressing release valve 704 to that air can escape main body 701 via base 702 at release port 703. In one embodiment, sensor 700 is connected to a cable 707 and an adaptor 708.

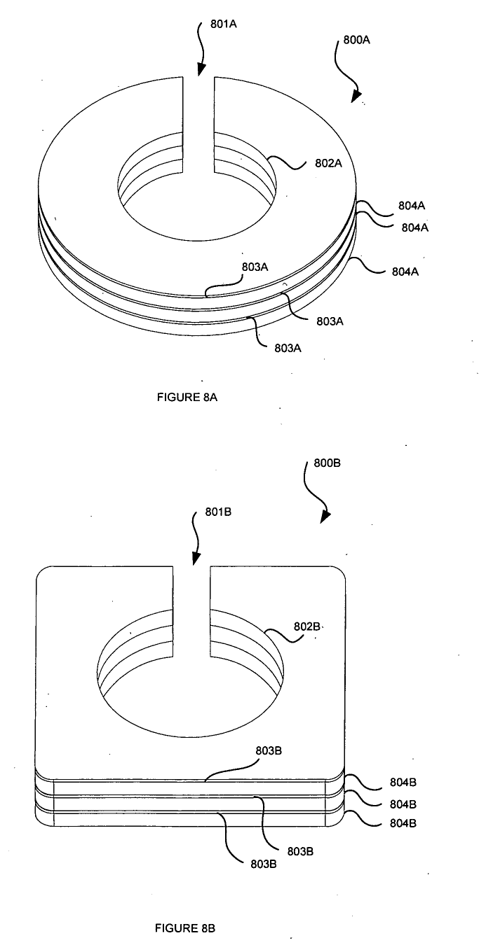

Referring to FIG. 8A, a cushion pad 800A that has a side cut 801A adapted to slide into a connector pin as to occupy a position between a sensor and a belt in accordance with an embodiment of the present invention is illustrated. In this embodiment, cushion pad 800A is a multi-layered pad that has first layers 803A and second layers 804A. In one embodiment, each second layer 804A is interposed between two adjacent first layers 803A. First layers 803A and second layers 804A have a concentric opening 802A that is connect to side cut 802A. Side cut 802A is adapted to slide to a connector pin described above so that cushion pad 800A can be positioned between a sensor and a belt as described above.

Continuing to FIG. 8B, a cushion pad 800B having a square shape that includes a side cut 801B adapted to slide into a connector pin so as to occupy a position between a sensor and a belt in accordance with an embodiment of the present invention is illustrated. Cushion pad 800B is similar in structure to cushion pad 800A but has a different geometrical shape. FIG. 8A and FIG. 8B show that cushion pad in accordance with the present invention can have any shape and structure so that cushion pad of the present invention can be positioned between a sensor and a holding device such as a belt, operable to press the sensor closer to the body of a patient without tightening the belt.

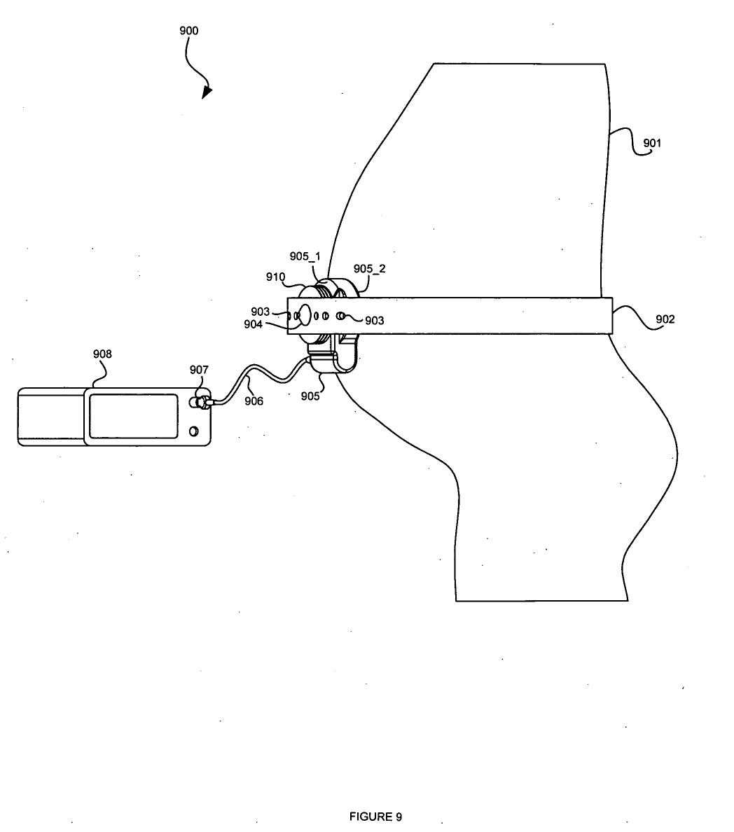

Now referring to FIG. 9, the operation of monitoring device 910 of the present invention as described above is illustrated. In operation, after the position of the fetus is located by a nurse, sensor 905 is attached the abdomen of patient 901 using a belt 902. More specifically, connector pin 904 is inserted in one of holes 903 and then belt 902 is wrapped around the abdomen of patient 901. Thereafter, connector pin 904 is again inserted into another hole 903 to hold sensor 905 in place where the fetus is. It is understood to a person skill in the art that different kind of belts that do not have holes may be used in place of belt 902. An electrical cable 906 is connected between sensor 905 and electronic display unit 908. A connector 907 can be used to connect cable 906 to electronic display unit 908. When vital signals cannot be detected due to different physical conditions of patient 901, a cushion pad 910 is inserted between belt 902 and sensor 905. The thickness of cushion pad 810 can be adjusted by cushion pads 230, 400, 500, 600, 700, 800A, and 800B described in FIG. 3 to FIG. 8A and FIG. 8B above.

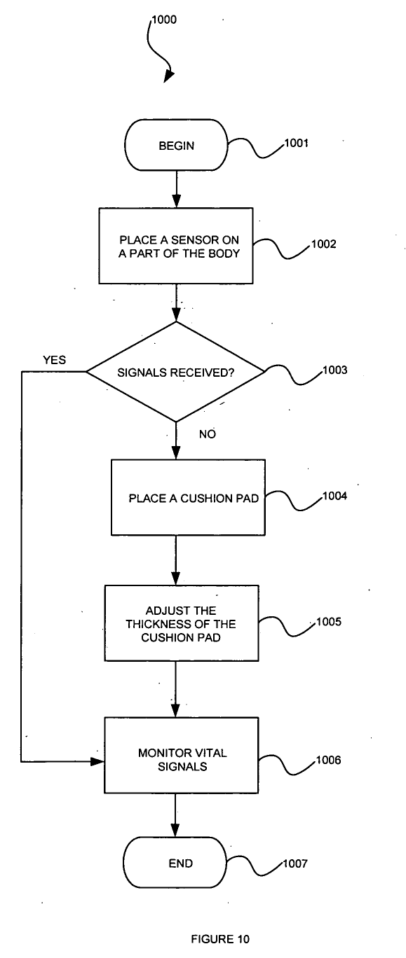

Now referring to FIG. 10, a flow chart 1000 showing a method of monitoring vital signals from the body of a patient is described. Method 1000 includes the steps of placing a sensor on the of a human body where vital signals to be acquired, determining whether vital signals are received, whenever vitals signals are not received, placing a cushion pad on the other side of the sensor; and adjusting the thickness of the cushion pad until the vitals signals are detected.

More particularly, the monitoring vital signals of a patient begins at step 1001.

Next, referring to step 1002, a sensor is placed on the body of a patient where vital signals are to be acquired. Step 1002 may also include determining the exact location on the body where vital signals are to be acquired using medical procedures. For example, in external fetal heart rate monitoring, the position of a fetus is first determined using medical procedures known as Leopold's techniques. Then, a sensor is placed where the fetus is best located. Referring to step 1003, determining whether vital signals are detected. In many instances, vital signals cannot be detected due to the physical conditions of a patient.

Referring next to step 1004 when vital signals are not detected, a cushion pad is inserted between the sensor and a belt so as to press the sensor closer to the body of the patient. Step 1004 can be implemented using cushion pads 230, 400, 500, 600, 700, 800A, and 800B described above. Then referring to step 1005, the thickness of the cushion pad is adjusted until vital signals are detected. In practice, step 1005 can be implemented using cushion pads 300 and 400 where layers can be removed, cushion pad 500 where electrical motor 504 is used, or cushion pad 600 where first cylindrical part 601_1 is mechanically twisted into second cylindrical part 601_2. In one embodiment, the thickness of cushion pad can be adjusted by manually pumping air into cushion pad 701 using hand pump 705.

Finally, referring to step 1006, vital signals are monitored until external monitoring ends at step 1007.

Obviously many modifications and variations of the present invention are possible in light of the above teachings. It is therefore to be understood that within the scope of the appended claims the invention may be practiced otherwise than as specifically described. It should be understood, of course, the foregoing disclosure relates only to a preferred embodiment (or embodiments) of the invention and that numerous modifications may be made therein without departing from the spirit and the scope of the invention as set forth in the appended claims. Various modifications are contemplated and they obviously will be resorted to by those skilled in the art without departing from the spirit and the scope of the invention as hereinafter defined by the appended claims as only a preferred embodiment(s) thereof has been disclosed.

What is claimed is:

1. A monitoring device, comprising:
a sensor having a front side and a back side, said front side being placed directly onto the body of a patient so as to acquire medical vital signals from within the body of said patient, said back side having a connector pin;
a belt operable to hold said sensor to a fixed location on a part of the body of said patient; and
a cushion pad, positioned between said sensor and said belt, operable to press said sensor close to said portion of the body of said patient.

2. The monitoring device of claim 1 wherein the thickness of said cushion pad is adjustable depending on the physical condition of said body of said patient.

3. The monitoring device of claim 2 wherein said cushion pad further comprises a plurality of removable layers.

4. The monitoring device of claim 3 wherein said cushion pad further comprises intermediate layers, each located between two adjacent removable layers of said cushion pad.

5. The monitoring device of claim 4 wherein said intermediate layer is made of non-adhesive material.

7. The monitoring device of claim 4 wherein the last layer that directly contacts said sensor is an adhesive layer.

8. The monitoring device of claim 1 further comprises an electrical cable electrically coupling said sensor to an electronic display device for displaying said vital signals from within said body of said patient.

9. The monitoring device of claim 2 wherein said the thickness of said cushion pad is adjustable by pumping air thereinto by a healthcare provider.

10. The monitoring device of claim 2 wherein said cushion pad further comprises a first portion and a second portion, each having threads adapted to facilitating the mechanical coupling of said first portion to said second portion adjustable by mechanically twisting said first portion onto said second portion by a healthcare provider so as to adjust the thickness of said cushion pad.

11. The monitoring device of claim 2 wherein said cushion pad further comprises:
a first portion mechanically coupled to an electrical motor controlled by a switch; and
a second portion mechanically coupled to said first portion so that whenever said switch is depressed by a healthcare provider, said electrical motor is operable to move said second portion vertically relative to said first portion so that the total thickness of said cushion is adjustable.

12. A method of measuring vital signals from within a human body, comprising:
placing a sensor on a portion of said human body;
determining whether vital signals are received;
whenever said vitals signals are not received, placing a cushion pad on the other side of said sensor; and
adjusting the thickness of said cushion pad until said vitals signals are received.

13. The method of claim 12 wherein said placing a sensor on said human body further comprises:
determining a location on said human body where vital signals are to be acquired using medical procedures; and
using an elastic belt to hold said sensor in place by securing a hole of said elastic belt onto a connector pin of said sensor.

14. The method of claim 13 wherein said adjusting the thickness of said cushion pad further comprises removing layers of said cushion pad.

15. The method of claim 12 wherein said adjusting the thickness of said cushion pad further comprises pumping air into said cushion pad.

16. The method of claim 12 wherein said adjusting the thickness of said cushion pad further comprises mechanically twisting a first portion and a second portion of said cushion pad together.

17. A device for improving the receipt of vital signals from within a body of a patient using an external monitoring device including a belt and a sensor, comprising:
means, inserted between said belt and said sensor, for pressing said sensor sufficiently close to said body of said patient without having to tighten said belt so as to better receive said vital signals without causing pains to said patient.

18. The device of claim 17 wherein said pressing means further comprises:
a plurality of removable layers, each adapted to be removed for adjusting the thickness of said pressing means; and
a plurality of non-adhesive intermediate layers, each being placed between two adjacent removable layers.

19. The device of claim 18 wherein each of said plurality of removable layers and said non-adhesive intermediate layers further comprises concentric hole for inserting into a button connector of a monitor.

20. The device of claim 28 wherein each of said plurality of removable layers and said non-adhesive intermediate layers further comprises a side cut for inserting into a button connector of a monitor.

21. The device of claim 17 wherein said pressing means further comprises:
a first portion and a second portion, both having threads so that said first portion is adapted to be screwed onto said second portion so as to adjust the thickness of said pressing means.

22. The device of claim 17 wherein said pressing means further comprises:
a first portion mechanically coupled to an electrical motor controlled by a switch; and
a second portion mechanically coupled to said first portion so that whenever said switch is depressed by a healthcare provider, said electrical motor is operable to move said second portion vertically relative to said first portion so that the total thickness of said pressing means is adjustable.

23. The device of claim 17 wherein said pressing means further comprises an air pump connected to said pressing means so that the thickness of said pressing means is adjustable by a healthcare provider's pumping air into said pressing means using said air pump.
